(19) United States
(12) Patent Application Publication
(10) Pub. No.: US 2019/0321407 A1
(43) Pub. Date: Oct. 24, 2019

(54) COMPOSITIONS AND METHODS FOR PREPARATION OF RED BLOOD CELLS

(71) Applicant: Cerus Corporation, Concord, CA (US)

(72) Inventors: Anna ERICKSON, Concord, CA (US); Mary Ann SCHOTT, Concord, CA (US); Blake WARBINGTON, Concord (US)

(21) Appl. No.: 16/474,938

(22) PCT Filed: Dec. 28, 2017
(86) PCT No.: PCT/US2017/068689

§ 371 Date: Jun. 28, 2019

Related U.S. Application Data
(60) Provisional application No. 62/441,312, filed on Dec. 31, 2016.

(51) Int. Cl. A61K 35/18; A61K 35/16; A61K 31/473; A61K 31/198; A61K 38/06

(52) U.S. Cl. CPC A61K 35/18; A61K 35/16; A61K 38/063; A61K 31/198; A61K 31/473

(57) ABSTRACT

The present disclosure relates to compositions and methods for treating red blood cells compositions with a pathogen-inactivation compound and for preparing pathogen-inactivated red blood cells.

### CROSS-REFERENCE TO RELATED APPLICATION

This application claims the priority benefit of U.S. Provisional Application Ser. No. 62/441,312, filed Dec. 31, 2016, which is incorporated herein by reference in its entirety.

### TECHNICAL FIELD

The present disclosure relates to compositions and methods for treating red blood cells compositions with a pathogen-inactivation compound and for preparing pathogen-inactivated red blood cells.

### BACKGROUND

The transfusion of blood or blood components is a commonly used and important practice for treating a variety of medical conditions. While the safety of blood products has improved through the years due to advances in blood donor screening and blood testing, the transmission of infectious disease by blood products remains a serious risk. In certain cases, such as the viruses HIV, HCV and HBV for which highly sensitive detection technologies are available, infections may nevertheless escape detection due to low levels of virus, such as during the period shortly after infection. In addition, detection assays are not available for many recognized infectious agents, along with risk of previously unknown or uncharacterized “emerging” pathogens.

Chemical agents have been introduced into blood or blood products to inactivate pathogens and potentially reduce or eliminate the need for screening technologies. For plasma and platelet products having minimal red blood cell content, photochemical (e.g., photoactivated) compounds such as psoralens (e.g., amotosalen with UVA) may be used for pathogen inactivation (see e.g., U.S. Pat. No. 5,593,823), and certain such technologies have received regulatory approval in many countries throughout the world. For red blood cell-containing blood products, nucleic acid targeting compounds are being developed for pathogen inactivation, which do not require photoactivation (see e.g., U.S. Pat. Nos. 6,093,725 and 6,514,987), and one such compound, S-303, has completed successful Phase 3 clinical testing (INTERCEPT® Blood System for Red Blood Cells, Cerus Corp.). The S-303 pathogen inactivation technology utilizes a disposable processing set that includes the pathogen-inactivation compound reconstituted in saline, together with a quencher, glutathione (see e.g., U.S. Pat. No. 8,900,805), also reconstituted in saline, for treating red blood cell (RBC) units. RBC units for pathogen inactivation may comprise, for example, about 110 mL RBC content in a total unit volume of about 220 mL (inclusive of any donor plasma, anticoagulant and additive solution), up to about 220 mL or even 250 mL RBC content in a total unit volume up to about 360 mL, inclusive of any plasma, anticoagulant and additive solution.

RBC donations collected with automated blood collection devices, such as for example, apheresis devices, provide an opportunity for collecting larger amounts of RBC's from a single donor compared to manual, whole blood collection methods. Double dose RBC donations may comprise, for example, about 220 mL up to or exceeding about 420 mL RBC content (plus additional volume from any donor plasma, anticoagulant and additive solution), which subsequently may be divided to yield two RBC units from a single donor versus a standard one unit collection. However, larger RBC collections, such as with double dose RBC collections, and associated increases in volumes for such collections, may limit processing of the RBC's for pathogen inactivation. Such larger volumes may be addressed in part by processing methods with more concentrated RBC units (e.g., units with higher hematocrit, units without RBC additive solution) as described in the present application. However, such methods also may result in a loss of beneficial properties arising from the inclusion of RBC additive solution.

There remains a need for improved red blood cell pathogen inactivation technologies, such as the S-303 based technology, which can be used for the treatment of larger amounts of RBC's, including for example, double dose red blood cell donations (e.g., double apheresis red blood cell donations). The present disclosure provides methods of treating larger RBC compositions (e.g., larger amounts of RBC's), including double dose RBC donations, particularly RBCs without the benefit of suspension in additive solutions.

### SUMMARY

The present disclosure provides compositions and methods for treating red blood cell compositions with a pathogen-inactivation compound and preparing pathogen-inactivated red blood cells. In particular, the compositions and methods, and related processing sets and kits provided herein, are particularly useful for treating and preparing red blood cells from double red blood cell donations.

In one aspect, the present disclosure provides method of treating a red blood cell composition, comprising: (a) mixing (i) a red blood cell composition comprising a double red blood cell donation (e.g., composition comprising a double donation of red blood cells obtained from a donor (e.g., one donor), double dose red blood cell donation; double red blood cell collection); (ii) an effective amount of a pathogen-inactivating compound comprising a functional group which is, or which forms, a reactive electrophilic group; and (iii) an effective amount of a quencher; in a processing solution (e.g., treatment solution, diluent solution); wherein at least one of the pathogen-inactivating compound and the quencher is in (e.g., suspended in, reconstituted in) a solution comprising dextrose (e.g., about 5% to about 12% dextrose, about 7% to about 12% dextrose, about 10% dextrose); (b) replacing the solution used during treatment of the red blood cell composition in step (a) with a red blood cell additive solution, to yield a pathogen-inactivated red blood cell preparation; and (c) separating (e.g., transferring a portion of) the pathogen-inactivated red blood preparation of step (b) into at least two units of pathogen-inactivated red blood cells suitable for infusion into a subject (e.g., pathogen-inactivated red blood cell products suitable for infusion), wherein each unit is contained in an individual container (e.g., storage container, storage bag). In some embodiments, the double red blood cell donation is obtained from a donor by apheresis. In some embodiments, the red blood cell composition comprises packed red blood cells. In some embodiments, the red blood cell composition comprises a packed cell volume of about 75% to about 95% or about 80% to about 90%. In some embodiments, the red blood cell composition comprises a red cell volume of at least about 220 mL, at least about 240 mL, at least about 260 mL, at least about 280 mL, at least about 300 mL, at least about 320 mL, at least about 340 mL, at least about 360 mL, at least about 380 mL, at least about 400 mL, at least about 420 mL or more. In some embodiments, the red blood cell composition comprises a red cell volume of less than about 450 mL, less than about 420 mL, less than about 400 mL, less than about 380 mL, or less than or equal to 360 mL. In some embodiments, the red blood cell composition comprises a red cell volume of about 220 mL to about 450 mL, about 220 mL to about 420 mL, about 220 mL to about 400 mL, about 220 mL to about 380 mL, about 220 mL to about 360 mL, about 240 mL to about 450 mL, about 240 mL to about 420 mL, about 240 mL to about 400 mL, about 240 mL to about 380 mL, about 240 mL to about 360 mL, about 260 mL to about 450 mL, about 260 mL to about 420 mL, about 260 mL to about 400 mL, about 260 mL to about 380 mL, about 260 mL to about 360 mL, about 280 mL to about 450 mL, about 280 mL to about 420 mL, about 280 mL to about 400 mL, about 280 mL to about 380 mL, about 280 mL to about 360 mL, or about 300 mL to about 360 mL. In some embodiments, the red blood cell composition comprises a red cell volume of about 220 mL, about 240 mL, about 260 mL, about 280 mL, about 300 mL, about 320 mL, about 340 mL, about 360 mL, about 380 mL, about 400 mL, or about 420 mL. In some embodiments, the red cell volume is an absolute RBC mass volume.

In some embodiments, the red blood cell composition further comprises plasma (e.g., residual donor plasma). In some embodiments, the red blood cell composition comprises less than about 20%, less than about, 15%, less than about 10%, or less than about 5% plasma by volume (e.g., percent of total volume). In some embodiments, the red blood cell composition comprises at least about 1%, at least about 3% or at least about 5% plasma by volume (e.g., percent of total volume). In some embodiments, the red blood cell composition comprises about 1% to about 20%, about 1% to about 15%, about 1% to about 10%, about 1% to about 5%, or about 5% to about 10% plasma by volume (e.g., percent of total volume). In some embodiments, the red blood cell composition further comprises an anticoagulant solution (e.g., ACD, ACD-A). In some embodiments, the red blood cell composition does not contain a red blood cell additive solution.

In some embodiments, the pathogen-inactivating compound comprises a nucleic acid binding ligand that is an intercalator. In some embodiments, the intercalator is an acridine. In some embodiments, the pathogen-inactivating compound is β-alanine, N-(acridin-9-yl), 2-[bis(2-chloroethyl)amino]ethyl ester. In some embodiments, the quencher comprises cysteine or a derivative of cysteine. In some embodiments, the quencher is a peptide of 3-6 amino acids, wherein at least one of the amino acids is cysteine, N-acetyl cysteine, or S-acetyl cysteine. In some embodiments, the quencher is glutathione or a pharmaceutically acceptable salt thereof. In some embodiments, the quencher is glutathione monosodium salt.

In some embodiments, at least one of the pathogen-inactivating compound and the quencher is in a solution (e.g., suspended in a solution, re-suspended in a solution) comprising about 5% to about 15%, about 5% to about 12%, about 5% to about 10%, about 7% to about 12%, or about 5%, about 6%, about 7%, about 8%, about 9%, about 10%, about 11% or about 12% dextrose (e.g., dextrose in water, dextrose in saline, dextrose in suitable buffer). In some embodiments, at least one of the pathogen-inactivating compound and the quencher is in a solution comprising about 5% to about 12% (e.g., 5-10%, 7-12%) dextrose in water. In some embodiments, at least one of the pathogen-inactivating compound and the quencher is in a solution comprising about 5% to about 12% (e.g., 5-10%, 7-12%) dextrose in saline. In some embodiments, only one of the pathogen-inactivating compound and the quencher is in a solution comprising dextrose (e.g., 5-12% dextrose, 5-10% dextrose, 7-12% dextrose). In some embodiments, each of the pathogen-inactivating compound and the quencher is in a solution comprising dextrose (e.g., 5-12% dextrose, 5-10% dextrose, 7-12% dextrose). In any or all of the aforementioned embodiments the percent dextrose is calculated as a weight/volume concentration. In some embodiments, the processing solution comprises one or more of dextrose, adenine, mannitol, citrate, citric acid, and phosphate. In some embodiments, the processing solution comprises one or more of adenine, mannitol, citrate, and phosphate.

In some embodiments, the method further comprises incubating the mixture of step (a) prior to the replacement of solution in step (b). In some embodiments, the mixture is incubated at room temperature (e.g., about 20° C. to about 25° C.). In some embodiments, the mixture is incubated for at least 2 hours, at least 6 hours, at least 12 hours, or at least 18 hours. In some embodiments, the mixture is incubated for 24 hours or less. In some embodiments, the mixture is incubated for about 2 hours to about 24 hours, about 6 hours to about 24 hours, about 12 hours to about 24 hours, or about 18 hours to about 24 hours. In some embodiments, the pathogen-inactivated red blood cell composition of step (b) is separated into two units of pathogen-inactivated red blood cells suitable for infusion into a subject. In some embodiments, the at least two units of pathogen-inactivated red blood cells suitable for infusion into a subject each comprise a therapeutic dosage unit of red blood cells. In some embodiments, the method further comprises storing the at least two units of pathogen-inactivated red blood cells at refrigerated temperature (e.g., 1° C. to 6° C.).

In another aspect, the present disclosure provides a unit of pathogen-inactivated red blood cells suitable for infusion into a subject prepared according to any of the aforementioned methods.

In another aspect, the present disclosure provides a method of infusing red blood cells into a subject in need thereof, comprising infusing into the subject an aforementioned unit of pathogen-inactivated red blood cells.

In another aspect, the present disclosure provides a method of preparing pathogen inactivated red blood cells from a double red blood cell donation, wherein the pathogen inactivated red blood cells are suitable for infusion into a subject, comprising: (a) collecting a double red blood cell donation from a donor by apheresis; (b) treating the red blood cells of the double red blood cell donation, by mixing (i) the red blood cells; (ii) an effective amount of a pathogen-inactivating compound comprising a functional group which is, or which forms, a reactive electrophilic group; and (iii) an effective amount of a quencher; in a processing solution; wherein at least one of the pathogen-inactivating compound and the quencher is in a solution comprising about 5% to about 12% dextrose (e.g., about 7% to about 12% dextrose); (c) replacing the solution used during treatment of the red blood cells in step (b) with a red blood cell additive solution to yield a pathogen-inactivated red blood cell preparation; and (d) separating the pathogen-inactivated red blood cell preparation of step (c) into at least two units of pathogen-inactivated red blood cells suitable for infusion into a subject, wherein each unit is contained in an individual container. In some embodiments, the red blood cells (e.g., red blood cells of the double red blood cell donation) comprise packed red blood cells. In some embodiments, the red blood cells comprise a packed cell volume of about 75% to about 95% or about 80% to about 90%. In some embodiments, the red blood cells (e.g., red blood cell composition) comprise a red cell volume of at least about 220 mL, at least about 240 mL, at least about 260 mL, at least about 280 mL, at least about 300 mL, at least about 320 mL, at least about 340 mL, at least about 360 mL, at least about 380 mL, at least about 400 mL, at least about 420 mL or more. In some embodiments, the red blood cells comprise a red cell volume of less than about 450 mL, less than about 420 mL, less than about 400 mL, less than about 380 mL, or less than or equal to 360 mL. In some embodiments, the red blood cells comprises a red cell volume of about 220 mL to about 450 mL, about 220 mL to about 420 mL, about 220 mL to about 400 mL, about 220 mL to about 380 mL, about 220 mL to about 360 mL, about 240 mL to about 450 mL, about 240 mL to about 420 mL, about 240 mL to about 400 mL, about 240 mL to about 380 mL, about 240 mL to about 360 mL, about 260 mL to about 450 mL, about 260 mL to about 420 mL, about 260 mL to about 400 mL, about 260 mL to about 380 mL, about 260 mL to about 360 mL, about 280 mL to about 450 mL, about 280 mL to about 420 mL, about 280 mL to about 400 mL, about 280 mL to about 380 mL, about 280 mL to about 360 mL, or about 300 mL to about 360 mL. In some embodiments, the red blood cells comprise a red cell volume of about 220 mL, about 240 mL, about 260 mL, about 280 mL, about 300 mL, about 320 mL, about 340 mL, about 360 mL, about 380 mL, about 400 mL, or about 420 mL. In some embodiments, the red cell volume is an absolute RBC mass volume.

In some embodiments, the red blood cells (e.g., red blood cells of the double red blood cell donation) further comprise plasma (e.g., residual donor plasma). In some embodiments, the red blood cells comprise less than about 20%, less than about, 15%, less than about 10%, or less than about 5% plasma by volume (e.g., percent of total volume). In some embodiments, the red blood cells comprise at least about 1%, at least about 3% or at least about 5% plasma by volume (e.g., percent of total volume). In some embodiments, the red blood cells comprise about 1% to about 20%, about 1% to about 15%, about 1% to about 10%, about 1% to about 5%, or about 5% to about 10% plasma by volume (e.g., percent of total volume). In some embodiments, the red blood cells further comprise an anticoagulant solution (e.g., ACD, ACD-A). In some embodiments, the red blood cells do not contain a red blood cell additive solution.

In some embodiments, the pathogen-inactivating compound comprises a nucleic acid binding ligand that is an intercalator. In some embodiments, the intercalator is an acridine. In some embodiments, the pathogen-inactivating compound is β-alanine, N-(acridin-9-yl), 2-[bis(2-chloroethyl)amino]ethyl ester. In some embodiments, the quencher comprises cysteine or a derivative of cysteine. In some embodiments, the quencher is a peptide of 3-6 amino acids, wherein at least one of the amino acids is cysteine, N-acetyl cysteine, or S-acetyl cysteine. In some embodiments, the quencher is glutathione or a pharmaceutically acceptable salt thereof. In some embodiments, the quencher is glutathione monosodium salt.

In some embodiments, at least one of the pathogen-inactivating compound and the quencher is in a solution (e.g., suspended in a solution, re-suspended in a solution) comprising about 5% to about 15%, about 5% to about 10%, about 5% to about 12%, about 7% to about 12%, or about 5%, about 6%, about 7%, about 8%, about 9%, about 10%, about 11% or about 12% dextrose (e.g., dextrose in water, dextrose in saline). In some embodiments, at least one of the pathogen-inactivating compound and the quencher is in a solution comprising about 5% to about 12% (e.g., 5-10%, 7-12%) dextrose in water. In some embodiments, at least one of the pathogen-inactivating compound and the quencher is in a solution comprising about 5% to about 12% (e.g., 5-10%, 7-12%) dextrose in saline. In some embodiments, only one of the pathogen-inactivating compound and the quencher is in a solution comprising dextrose (e.g., 5-12% dextrose, 5-10% dextrose, 7-12% dextrose). In some embodiments, each of the pathogen-inactivating compound and the quencher is in a solution comprising dextrose (e.g., 5-12% dextrose, 5-10% dextrose, 7-12% dextrose). In some embodiments, the processing solution comprises one or more of dextrose, adenine, mannitol, citrate, citric acid, and phosphate. In some embodiments, the processing solution comprises one or more of adenine, mannitol, citrate, and phosphate.

In some embodiments, the method further comprises incubating the mixture of step (b) prior to the replacement of solution in step (c). In some embodiments, the mixture is incubated at room temperature (e.g., about 20° C. to about 25° C.). In some embodiments, the mixture is incubated for at least 2 hours, at least 6 hours, at least 12 hours, or at least 18 hours. In some embodiments, the mixture is incubated for 24 hours or less. In some embodiments, the mixture is incubated for about 2 hours to about 24 hours, about 6 hours to about 24 hours, about 12 hours to about 24 hours, or about 18 hours to about 24 hours. In some embodiments, the pathogen-inactivated red blood cell composition of step (c) is separated into two units of pathogen-inactivated red blood cells suitable for infusion into a subject. In some embodiments, the at least two units of pathogen-inactivated red blood cells suitable for infusion into a subject each comprise a therapeutic dosage unit of red blood cells. In some embodiments, the method further comprises storing the at least two units of pathogen-inactivated red blood cells at refrigerated temperature (e.g., 1° C. to 6° C.).

In another aspect, the present disclosure provides a unit of pathogen-inactivated red blood cells suitable for infusion into a subject prepared according to any of the aforementioned methods.

In another aspect, the present disclosure provides a method of infusing red blood cells into a subject in need thereof, comprising infusing into the subject an aforementioned unit of pathogen-inactivated red blood cells.

### DESCRIPTION OF THE DRAWINGS

FIG. 1A shows an exemplary processing set for use in preparing pathogen inactivated red blood cell units in accordance with some embodiments. Dotted components depict 1) a second storage container for pathogen inactivated RBCs and associated tubing for connecting two storage containers, which may be integrated with (e.g., connected as part of) the processing set and/or coupled (e.g., sterile connected) to the processing set, and 2) a container of RBCs to be treated after coupling (e.g., by sterile connection) to the processing kit. Abbreviations: PIC, pathogen inactivating compound.

FIG. 1B shows an exemplary processing set for use in preparing pathogen inactivated red blood cell units in accordance with some embodiments. Provided with the processing set is a second storage container for pathogen inactivated RBCs and associated tubing configured to be coupled (e.g., sterile connected) with the rest of the processing set. Abbreviations: PIC, pathogen inactivating compound.

FIG. 1C shows an exemplary processing set for use in preparing pathogen inactivated red blood cell units in accordance with some embodiments. Provided with the processing set is a second storage container for pathogen inactivated RBCs and associated tubing that is integrated with (e.g., connected as part of) the rest of the processing set. Abbreviations: PIC, pathogen inactivating compound.

FIG. 2A shows an exemplary processing kit for use in preparing pathogen inactivated red blood cell units in accordance with some embodiments. Dotted components depict 1) a second storage container for pathogen inactivated RBCs and associated tubing for connecting two storage containers, which may be integrated with (e.g., connected as part of) the processing set and/or coupled (e.g., sterile connected) to the processing set, and 2) a container of RBC to be treated after coupling (e.g., by sterile connection) to the processing kit. Abbreviations: PIC, pathogen inactivating compound.

FIG. 2B shows an exemplary processing kit for use in preparing pathogen inactivated red blood cell units in accordance with some embodiments. Dotted components depict 1) a second storage container for pathogen inactivated RBCs and associated tubing for connecting two storage containers, which may be integrated with (e.g., connected as part of) the processing set and/or coupled (e.g., sterile connected) to the processing set, and 2) a container of RBC to be treated after coupling (e.g., by sterile connection) to the processing kit. Further provided with the processing set are containers containing the pathogen inactivating compound and quencher, respectively, which are both configured to be coupled (e.g., sterile connected) with the rest of the processing set. Abbreviations: PIC, pathogen inactivating compound.

FIG. 2C shows an exemplary processing kit for use in preparing pathogen inactivated red blood cell units in accordance with some embodiments. Dotted components depict a container of RBC to be treated after coupling (e.g., by sterile connection) to the processing kit. Provided with the processing set is a second storage container for pathogen inactivated RBCs and associated tubing configured to be coupled (e.g., sterile connected) with the rest of the processing set. Further provided with the processing set are containers containing the pathogen inactivating compound and quencher, respectively, which are both configured to be coupled (e.g., sterile connected) with the rest of the processing set. Abbreviations: PIC, pathogen inactivating compound.

FIG. 2D shows an exemplary processing kit for use in preparing pathogen inactivated red blood cell units in accordance with some embodiments. Dotted components depict a container of RBC to be treated after coupling (e.g., by sterile connection) to the processing kit. Provided with the processing set is a second storage container for pathogen inactivated RBCs and associated tubing integrated with (e.g., connected as part of) the rest of the processing set. Further provided with the processing set are containers containing the pathogen inactivating compound and quencher, respectively, which are both configured to be coupled (e.g., sterile connected) with the rest of the processing set. Abbreviations: PIC, pathogen inactivating compound.

### DETAILED DESCRIPTION

The present disclosure provides compositions and methods for preparing pathogen-inactivated red blood cells. While treatment of red blood cells with a pathogen-inactivating compound has been described, e.g., with processing sets developed for single RBC units, we have found that such methods may not be particularly suited, for example, for the preparation of pathogen-inactivated red blood cells from larger RBC compositions (e.g., compositions with larger amounts of RBC's), such as for example from a double red blood cell donation. The present application provides improved processes and kits for RBC pathogen inactivation, including but not limited to treatment of RBC compositions with a lower overall volume of suspension relative to RBC volume (e.g., high hematocrit), which may in some embodiments not include any RBC additive solution. In particular, the improved processes and kits provided herein may include providing at least one of the pathogen-inactivating compound and quencher in a solution comprising dextrose. Improved osmolarity, glucose concentrations and/or ATP levels may benefit from the improved processes and kits provided herein, including from the provision of at least one of the pathogen-inactivating compound and quencher in a solution comprising dextrose. The compositions and methods, and related processing sets and kits of the present application are particularly useful for the effective preparation of pathogen-inactivated red blood cells from larger RBC compositions, including a double red blood cell donation, such as for example, from apheresis collection.

The articles “a” and “an” are used herein to refer to one or to more than one (i.e., to at least one) of the grammatical object of the article.

### Compositions

Provided herein are compositions and methods for treating a red blood cell composition with an effective amount of a pathogen-inactivating compound, such as, for example, red blood cell compositions not comprising (e.g., not including, without) additive solution. Non-limiting examples include compositions and methods for use in the preparation of pathogen-inactivated red blood cells obtained from a donor as a double red blood cell donation (e.g., double dose red blood cell collection, a double red blood cell apheresis collection).

### Red Blood Cells

Red blood cell (RBC) compositions of the present disclosure include, but are not limited to, any blood product comprising red blood cells (e.g., human blood), wherein the blood product provides, or is processed to provide, red blood cells suitable for use in humans, mammals, and/or vertebrates, such as for infusion. Red blood cell compositions include, for example, whole blood collected red blood cells, apheresis collected red blood cells and red blood cell concentrates, such as packed red blood cells (pRBCs; e.g., red blood cells with increased hematocrit and/or not containing additive solution). The red blood cell compositions may be described by their hematocrit or packed cell volume (PCV), a measure of the concentration of red blood cells in the composition. Red blood cell compositions may have a hematocrit (e.g., PCV) in the range of about 1 to 100%, more likely about 10 to 90%, also about 35 to 80%, or about 40 to 70%, or about 70% to about 90%. The hematocrit or PCV may, for example, may be used to describe the input red blood cell compositions subjected to the methods (e.g., treatment, preparation) provided herein and/or the red blood cells after subjecting the RBCs to the methods herein (e.g., unit of pathogen-inactivated red blood cells suitable for infusion). Such red blood cell compositions may include chemicals, such as pathogen-inactivating compounds and other components, such as for example, quenchers. They may also include buffers and other solutions, such as red blood cell additive solutions (e.g., any solution described in herein, such as SAG-M, AS-5 or any solution of Table 1), including salts or buffered solutions, anticoagulants (e.g., ACD, CPD, CP2D). They may also include plasma (e.g., residual donor plasma). In some preferred embodiments, red blood cell compositions to be subjected to a method of the present disclosure (e.g., pathogen inactivation) comprise plasma and/or anticoagulant, but no red blood cell additive solution.

In some embodiments, the red blood cell compositions described herein (e.g., for use in the methods described herein), such as for example, red blood cells obtained from a donor as a double red blood cell donation (e.g., double dose apheresis red blood cell collection), are red blood cells (e.g., packed red blood cells) having a hematocrit (e.g., PCV) in the range of about 70% to about 95%, about 70% to about 90%, about 70% to about 85%, about 70% to about 80%, about, about 75% to about 95%, about 75% to about 90%, about 75% to about 85%, about 80% to about 95%, about 80% to about 90%, or about 95%, about 90%, about 85%, about 80%, about 75%, or about 70%, prior to use in the methods (e.g., methods of treating, methods of preparing) described herein. In some embodiments, the red blood cell composition (e.g., double red blood cell donation) has a hematocrit of at least about 70%, at least about 75%, at least about 80%, at least about 85%, or at least about 90%. In some embodiments, the red blood cell composition (e.g., double red blood cell donation) is collected as a “dry” apheresis collection, such as for example with a minimal amount of plasma and/or anticoagulant (e.g., no additive solution). In some embodiments, the red blood cell composition (e.g., double red blood cell donation) described herein may comprise (e.g., comprise an RBC content of) at least about 220 mL RBC volume, at least about 230 mL RBC volume, at least about 240 mL RBC volume, at least about 250 mL RBC volume, at least about 260 mL RBC volume, at least about 270 mL RBC volume, at least about 280 mL RBC volume, at least about 290 mL RBC volume, at least about 300 mL RBC volume, at least about 310 mL RBC volume, at least about 320 mL RBC volume, at least about 330 mL RBC volume, at least about 340 mL RBC volume, at least about 350 mL RBC volume, at least about 360 mL RBC volume, at least about 370 mL RBC volume, at least about 380 mL RBC volume, at least about 390 mL RBC volume, or at least about 400 mL or more RBC volume. In some embodiments, the red blood cell composition (e.g., double red blood cell donation) may comprise about 220 mL to about 420 mL RBC volume, about 230 mL to about 420 mL RBC volume, about 240 mL to about 420 mL RBC volume, about 250 mL to about 420 mL RBC volume, about 220 mL to about 400 mL RBC volume, about 220 mL to about 390 mL RBC volume, about 220 mL to about 380 mL RBC volume, about 220 mL to about 370 mL RBC volume, about 220 mL to about 360 mL RBC volume, about 240 mL to about 400 mL RBC volume, about 240 mL to about 390 mL RBC volume, about 240 mL to about 380 mL RBC volume, about 240 mL to about 370 mL RBC volume, about 240 mL to about 360 mL RBC volume, about 260 mL to about 400 mL RBC volume, about 260 mL to about 390 mL RBC volume, about 260 mL to about 380 mL RBC volume, about 260 mL to about 370 mL RBC volume, about 260 mL to about 360 mL RBC volume, about 280 mL to about 400 mL RBC volume, about 280 mL to about 390 mL RBC volume, about 280 mL to about 380 mL RBC volume, about 280 mL to about 370 mL RBC volume, about 280 mL to about 360 mL RBC volume, about 300 mL to about 400 mL RBC volume, about 300 mL to about 390 mL RBC volume, about 300 mL to about 380 mL RBC volume, about 300 mL to about 370 mL RBC volume, or about 300 mL to about 360 mL RBC volume. In some embodiments, RBC compositions (e.g., packed red blood cells) for use as disclosed herein (e.g., input RBCs, prior to subjecting to treatment, prior to mixing with a pathogen-inactivating compound) may comprise plasma and/or anticoagulant, but without additive solution. Anticoagulants and useful volumes thereof are well known in the art. Double red blood cell donation (e.g., double dose RBC collection by apheresis) is well known in the art, such as by those in the blood collection, blood processing and/or transfusion medicine fields. Any of several commercially available blood collection devices (e.g., blood cell separators, apheresis devices) may be used for collecting a double red blood cell donation, including for example, the Trima Accel (TerumoBCT), Alyx (Fenwal), and MCS+(Haemonetics) devices.

In some embodiments, the red blood cell compositions are not packed red blood cells (e.g., non-packed RBCs), such as for example, RBC's having a hematocrit (e.g., PCV) in the range about 50% to about 70%, about 55% to about 70%, about 50% to about 65%, about 55% to 65%, or about 70%, about 65%, about 60%, about 55% or about 50%, during and/or at the end of (e.g., after) use in the methods described herein. In some embodiments, the red blood cells after treatment (e.g., pathogen-inactivated red blood cells; units of pathogen-inactivated red blood cells suitable for infusion), such as for example, after replacing the solution used during treatment with a final additive solution, have a hematocrit as set forth above for non-packed RBCs. In some embodiments, the red blood cell compositions are diluted with a diluent solution and have a hematocrit (e.g., PCV) in the range about 30 to 50%, or about 35 to 45%, or about 40%, during use in the methods of treating described herein. In some embodiments, the red blood cell compositions described herein have been leukoreduced prior to use in the methods (e.g., methods of treating and/or methods of preparing) described herein. In some embodiments, the red blood cell compositions have not been leukoreduced. Any red blood cell composition that will come into contact with, or be introduced into, a living human, mammal, or vertebrate, where such contact carries a risk of transmitting disease due to contaminating pathogens may be treated as disclosed herein.

Where the methods provided herein result in (e.g., yield) at least two units of pathogen-inactivated red blood cells suitable for infusion into a subject (e.g., in separate containers), each such unit may comprise (e.g., comprise an RBC content of) at least about 110 mL RBC volume, at least about 115 RBC volume, at least about 120 mL RBC volume, at least about 125 mL RBC volume, at least about 130 mL RBC volume, at least about 135 mL RBC volume, at least about 140 mL RBC volume, at least about 145 mL RBC volume, at least about 150 mL RBC volume, at least about 155 mL RBC volume, at least about 160 mL RBC volume, at least about 165 mL RBC volume, at least about 170 mL RBC volume, at least about 175 mL RBC volume, at least about 180 mL RBC volume, at least about 185 mL RBC volume, at least about 190 mL RBC volume, at least about 195 mL RBC volume, or at least about 200 mL RBC volume. In some embodiments, the RBC content and/or RBC volume is an average RBC content and/or RBC volume determined from among a plurality of units.

### Pathogens

A pathogen contaminant, if present, to be inactivated in the methods of the disclosure includes any nucleic acid-containing agent capable of causing disease in a human, other mammals, or vertebrates. The pathogenic agent may be unicellular or multicellular. Examples of pathogens are bacteria, viruses, protozoa, fungi, yeasts, molds, and mycoplasmas which cause disease in humans, other mammals, or vertebrates. The genetic material of the pathogen may be DNA or RNA, and the genetic material may be present as single-stranded or double-stranded nucleic acid. While pathogens known to cause disease are well known in the art, the disclosure also contemplates the inactivation of previously unrecognized pathogens (e.g., emerging pathogens), as well as pathogens not definitively associated as the causal agent of a disease. In some embodiments, the presence of contaminating leukocytes may also result in disease, and the present disclosure contemplates the inactivation of such leukocytes.

### Pathogen Inactivating Compounds

The inactivation of a pathogen in the red blood cell compositions is effected by contacting the pathogen in the red blood cell composition with a pathogen-inactivating compound. In any of the embodiments described herein, the pathogen-inactivating compound (e.g., S-303 described herein) may be present in an effective amount (e.g., an effective amount to inactivate a pathogen, such as an amount sufficient to inactivate, for example, at least 1 log, 2 log, 3 log, 4 log or more of a pathogen in the red blood cell composition, if present). Pathogen-inactivating compounds that may be used by the methods of the disclosure include compounds that comprise a functional group which is, or which is capable of forming and has formed, e.g. in situ, a reactive group, such as an electrophilic group. In some cases, the pathogen-inactivating compounds of the present disclosure do not require photoactivation to be reactive. For example, the functional group may be a mustard group, a mustard group intermediate, a mustard group equivalent, an epoxide, a formaldehyde or a formaldehyde synthon. Such functional groups are capable of forming in situ a reactive group, such as an electrophilic aziridine, aziridinium, thiirane or thiiranium ion. A mustard group may be a mono- or bis-(haloethyl)amine group or a mono (haloethyl)sulfide group. A mustard equivalent is a group that reacts by a mechanism similar to the mustards, for example by forming reactive intermediates such as aziridinium and aziridine groups or thiirane and thiiranium groups. Examples include aziridine derivatives, mono or bis-(mesylethyl)amine groups, mono-(mesylethyl)sulfide groups, mono or bis-(tosylethyl)amine groups and mono-(tosylethyl)sulfide groups. A formaldehyde synthon is any compound that breaks down to a formaldehyde, which includes a hydroxylamine such as hydroxymethylglycine. The reactive group of the pathogen-inactivating compound is capable of reacting with the nucleic acids of pathogens, for example with nucleophilic groups on the nucleic acid. The reactive group is also capable of reacting with a nucleophilic group of a quencher. Pathogen-inactivating compounds may also include a component that targets the compound to nucleic acids, such as an anchor portion. The anchor portion comprises a moiety which is capable of binding non-covalently to a nucleic acid biopolymer, such as DNA or RNA, and is also referred to as a nucleic acid binding ligand, nucleic acid binding group, or nucleic acid binding moiety. Examples of such compounds are described in U.S. Pat. Nos. 5,691,132, 6,410,219, 6,136,586, 6,617,157, and 6,709,810, each of which is incorporated by reference herein. Another class of pathogen-inactivating compounds that may be quenched by the methods of the disclosure comprises the above-mentioned reactive groups linked to a nucleic acid binding group via a hydrolysable linker, as described in U.S. Pat. No. 6,514,987, incorporated by reference herein. The anchor portion of the pathogen-inactivating compounds has an affinity for nucleic acids. This affinity may be due to any of several modes of binding to the nucleic acid non-covalently, including, but not limited to, intercalation, minor groove binding, major groove binding, and electrostatic binding (e.g., phosphate backbone binding). The affinity may also be due to mixed modes of binding (e.g., intercalation and minor groove binding). The binding may be sequence-specific (i.e., increased binding affinity for one or more particular nucleic acid sequences over other nucleic acid sequences) or non sequence-specific. Detailed examples of such nucleic acid binding moieties can be found in the above-mentioned patents.

In some embodiments of each of the methods, compositions, processing sets and kits described herein, the pathogen-inactivating compound may comprise a functional group which is, or which forms, a reactive electrophilic group reactive with the nucleophile of the chosen quencher. In some embodiments, the pathogen-inactivating group comprises a nucleic acid binding ligand and a functional group which is, or which forms an electrophilic group. A specific example of a suitable pathogen-inactivating compound for use in the present invention is β-alanine, N-(acridin-9-yl), 2-[bis(2-chloroethyl)aminolethy]ester (also alternatively referred to herein as “S-303”), the structure of which is described in the above-referenced patents, including salts thereof.

In some embodiments, the concentration of the pathogen-inactivating compound, such as for example S-303, in the mixture with the red blood cells (e.g., red blood cell composition) and the quencher is in the range of about 0.05 mM to 4 mM, about 0.05 mM to 2 mM, about 0.05 mM to 0.5 mM, about 0.1 mM to 0.3 mM, or about 0.2 mM. In some embodiments, the molar ratio of quencher to pathogen inactivation compound once both components have been mixed with the red blood cell composition is about 10:1 to about 400:1, also about 10:1 to about 200:1, also about 20:1 to about 200:1, also about 50:1 to about 200:1, also about 100:1.

### Quenchers

Quenchers for use in methods of the present disclosure are intended to reduce unwanted side-reactions of the reactive electrophilic species used to inactivate pathogens (e.g., binding of the pathogen-inactivating compound to the red blood cell surface which may lead to an undesired immune response). In any of the embodiments described herein, the quencher (e.g., glutathione described herein) may be present in an effective amount (for example, an effective amount to reduce unwanted side reactions, such as the amounts described herein). Suitable quenchers comprise a nucleophilic group that is capable of reacting with the electrophilic group of the pathogen-inactivating compound. Non-limiting examples are described in detail in U.S. Pat. No. 6,709,810, incorporated by reference herein in its entirety. In some embodiments, the quenchers are capable of significantly reducing the unwanted side reactions in a red blood cell composition while allowing the pathogen-inactivating compound to sufficiently inactivate a pathogen that may be contaminating the red blood cell composition. In some embodiments, the improved methods of the present invention provide an effective amount of quencher in combination with an effective amount of pathogen-inactivating compound under conditions which provide optimal reduction in unwanted side reactions combined (e.g., binding of the pathogen-inactivating compound) with sufficient inactivation of pathogens, without significantly altering (e.g., without decreasing) the cell osmotic fragility and without significantly altering (e.g., without increasing) dehydration. A variety of unwanted side reactions may be reduced, such as reaction of the pathogen-inactivating compound with proteins and/or red blood cell components. In some embodiments, the quencher provides optimal reduction in the modification of the red blood cells, such as the binding of IgG to the red blood cells or binding of the pathogen-inactivating compound to the red blood cells. While the methods provided involve the ex vivo treatment of red blood cells (e.g., red blood cell compositions), some quenchers may remain in the composition upon introduction into an individual. As such, in some embodiments, the quenchers of the disclosure are suitable for infusion. Suitable quenchers include, but are not limited to, compounds comprising a thiol group, such as quenchers comprising the amino acid cysteine or a suitable derivative of cysteine, such as N-acetyl cysteine. Examples of such quenchers include cysteine and peptides comprising at least one cysteine, such as glutathione. In some embodiments, the suitable quenchers comprise a derivative of cysteine that can form a thiol group in situ, with or without the use of additional chemicals or added enzymes, such as S-acetyl cysteine or other suitable thiol derived prodrugs of cysteine, or peptides comprising S-acetyl cysteine or other suitable thiol derived prodrugs of cysteine. Suitable derivatives of cysteine are those which either comprise, or are capable of forming in situ, a cysteinyl thiol which is capable of reacting with the electrophilic group of the pathogen-inactivating compound.

In some embodiments, the quencher is a peptide of 2 to 10 amino acids, wherein at least one of the amino acids is cysteine, N-acetyl cysteine, S-acetyl cysteine, or other suitable derivative of cysteine. In some embodiments, the quencher is a peptide of at least 3 amino acids, such as about 3-10 amino acids, also about 3-6 amino acids, wherein at least one of the amino acids is cysteine, N-acetyl cysteine, S-acetyl cysteine, or other suitable derivative of cysteine. In some embodiments, the quencher is a peptide of at least 3 amino acids, such as about 3-10 amino acids, also about 3-6 amino acids, wherein at least one of the amino acids is cysteine, N-acetyl cysteine, S-acetyl cysteine, or other suitable derivative of cysteine, also wherein at least 2 or at least 3 of the amino acids is cysteine, N-acetyl cysteine, S-acetyl cysteine, or other suitable derivative of cysteine.

In a preferred embodiment, the quencher is neutralized glutathione (also known as L-glutathione and γ-L-glutamyl-L-cysteinyl-glycine). Glutathione has many properties that make it particularly useful as a quencher. It is normally present in all cell types. It is not believed to be able to passively penetrate into a pathogen, such as by passing through cell membranes or lipid coats, of bacteria and lipid-enveloped viruses, or by passing through the viral capsid of non-enveloped viruses. At approximately neutral pH glutathione is charged and in the absence of active transport, does not penetrate lipid bilayers to any significant extent. Preferred methods of quenching are provided wherein contamination of a red blood cell composition by a viral or bacterial pathogen is inactivated by at least 2 log, preferably at least 3 log or 4 log or more. At the appropriate conditions, as described by the present disclosure, glutathione is also compatible with in vitro storage of red blood cells and the resulting red blood cell composition is suitable for introduction (e.g., infusion into a subject) in vivo.

In some embodiments, the quencher is glutathione in its reduced form. Glutathione disulfide, the oxidized form of glutathione, may also be used, so long as the glutathione disulfide is sufficiently reduced in solution prior to addition of the solution to the mixture comprising the red blood cells (e.g., red blood cell composition) or sufficiently reduced after addition to the mixture comprising the red blood cells.

In some embodiments, the quencher is a derivative of glutathione, such as a glutathione monoalkyl ester or dialkyl ester, wherein the alkyl group is a straight or branched group having 1 to 10 carbon atoms. Specific examples of alkyl groups include, but are not limited to methyl group, ethyl group, propyl group, isopropyl group, butyl group, isobutyl group, tert-butyl group, pentyl group, isopentyl group, neopentyl group, tert-pentyl group, 1-methylbutyl group, hexyl group, isohexyl group, 2-methylpentyl group, 1-ethylbutyl group, heptyl group, octyl group, nonyl group, and decyl group. For instance, non-limiting examples of glutathione derivatives include glutathione methyl ester, glutathione monoethyl ester, and glutathione monoisopropyl ester. In some embodiments, glutathione oxidized diethyl ester (GSSG-(glycyl)-diethyl-ester) is used. In some embodiments, a thioester of glutathione is hydrolyzed after addition to the red blood cell compositions to form the thiol.

It is understood that in some embodiments, the quencher will be provided in the form of a free acid or base, whereas, in other embodiments, the quencher will be provided in the form of a salt. If the quencher is in the form of a salt, the salt is preferably a pharmaceutically acceptable salt. The pharmaceutically-acceptable salts of compounds (in the form of water- or oil-soluble or dispersible products) include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-napthalensulfonate, nicotinate, oxalate, pamoate, pectinate, persulfate, 3-phenylpropionate, phosphate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, didbutyl; and diamyl sulfates, long chain halides such as decyl, lauryl, myristyl and stearyl chlorides, bromides and iodides, aralkyl halides like benzyl and phenethyl bromides and others. Other pharmaceutically acceptable salts include the sulfate salt ethanolate and sulfate salts.

For example, in some embodiments, the quencher is in the form of a pharmaceutically acceptable salt formed from glutathione. In some embodiments, the quencher is in the form of a pharmaceutically acceptable salt formed from glutathione and one or more cations such as sodium, aluminum, calcium, lithium, magnesium, zinc, or tetramethylammonium. In some embodiments, the quencher is glutathione (reduced) and is provided in the form of glutathione monosodium salt (available, e.g., from Biomedica Foscama, Italy). In some other embodiments, the glutathione (reduced) is provided in the form of glutathione hydrochloride salt. In some other embodiments, the glutathione is provided in the form of a glutathione (reduced) disodium salt. In further embodiments, a glutathione monoalkyl ester sulfate is used. In some embodiments, glutathione is provided in the form of glutathione oxidized disodium.

In some embodiments, the concentration of the quencher, such as for example glutathione, in the mixture with the red blood cells (e.g., red blood cell composition) and the pathogen inactivation compound is greater than 2 mM. In some embodiments, the quencher concentration is about 5 mM to about 30 mM. In some embodiments, the quencher concentration is about 15 mM to about 25 mM. In some embodiments, the quencher concentration is about 20 mM.

### Processing Sets and Kits

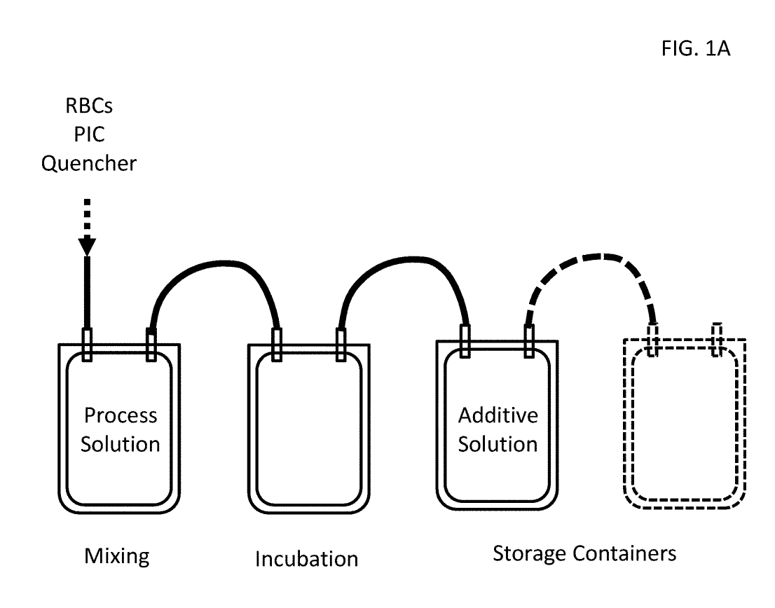

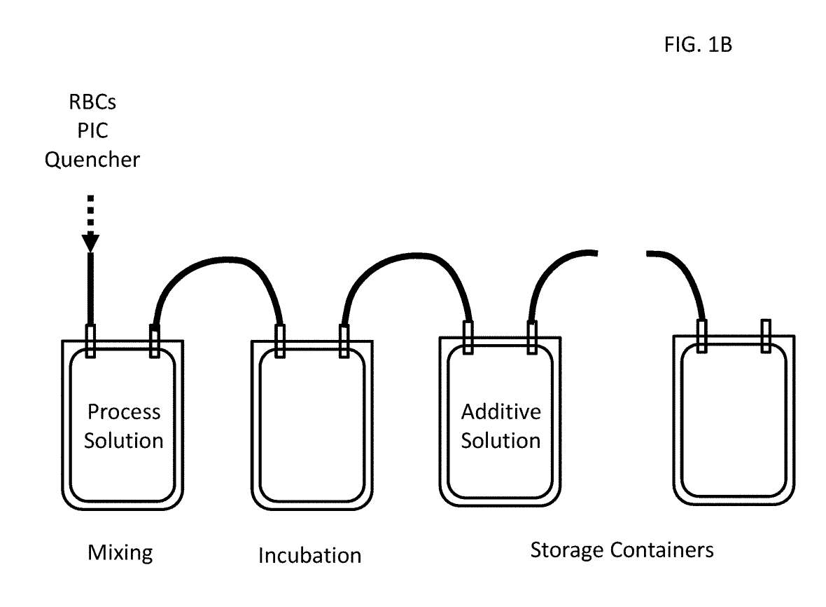

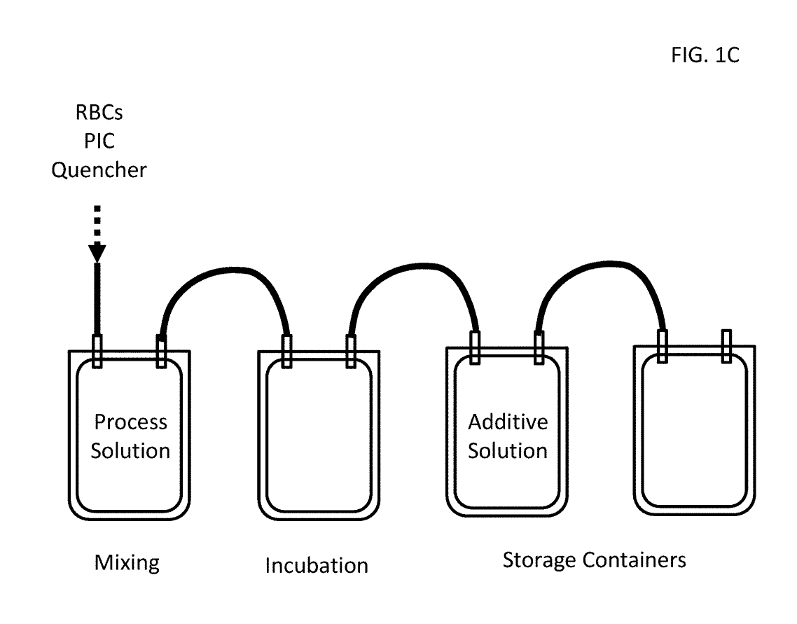

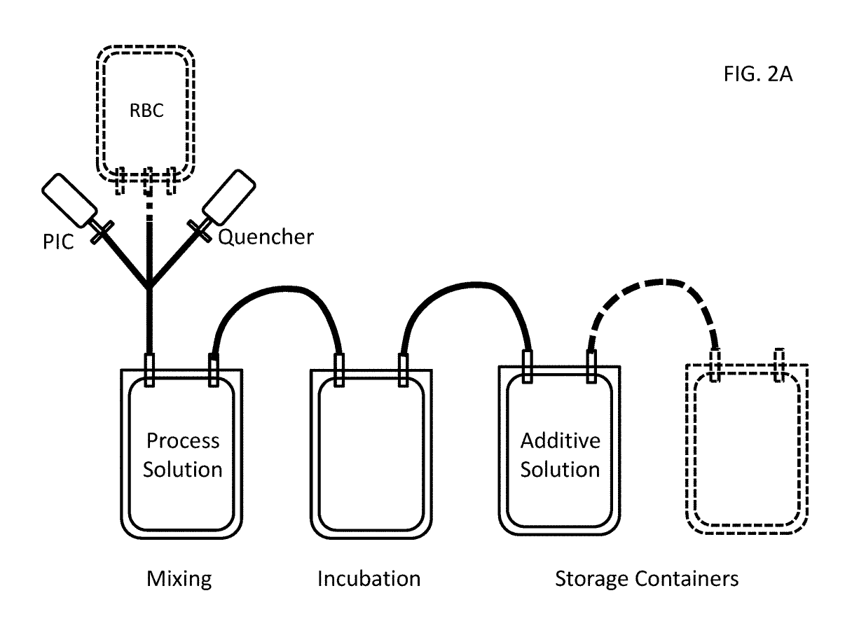

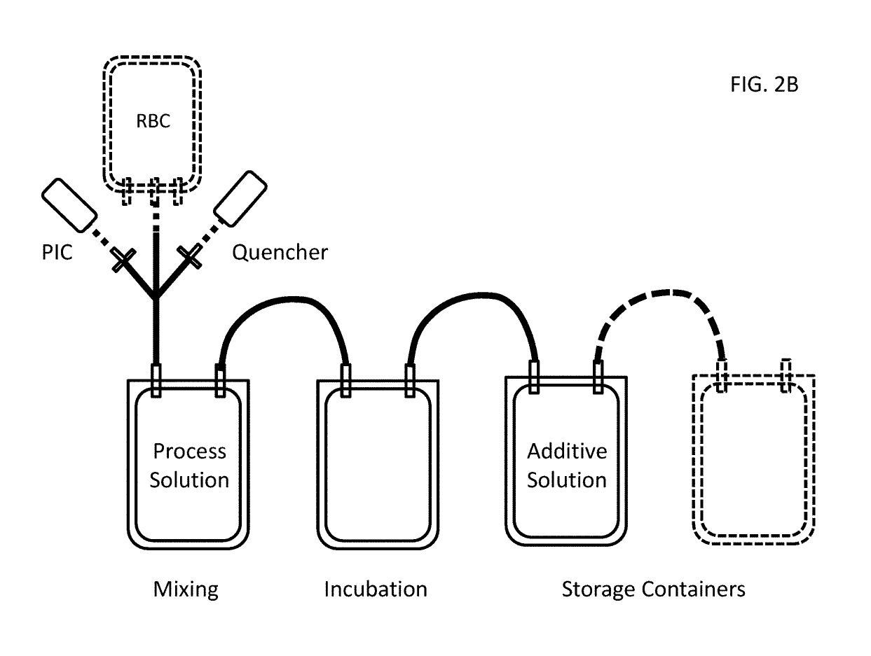

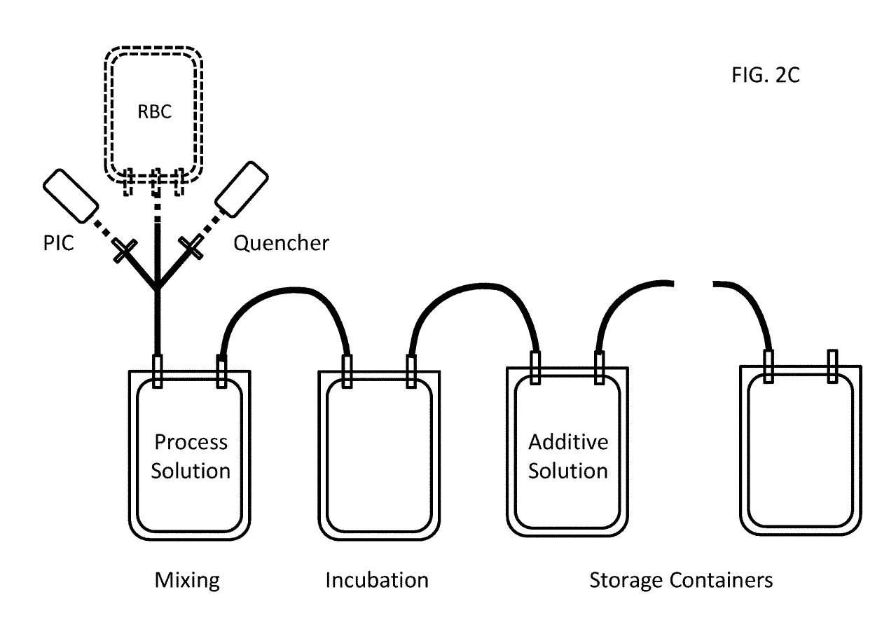

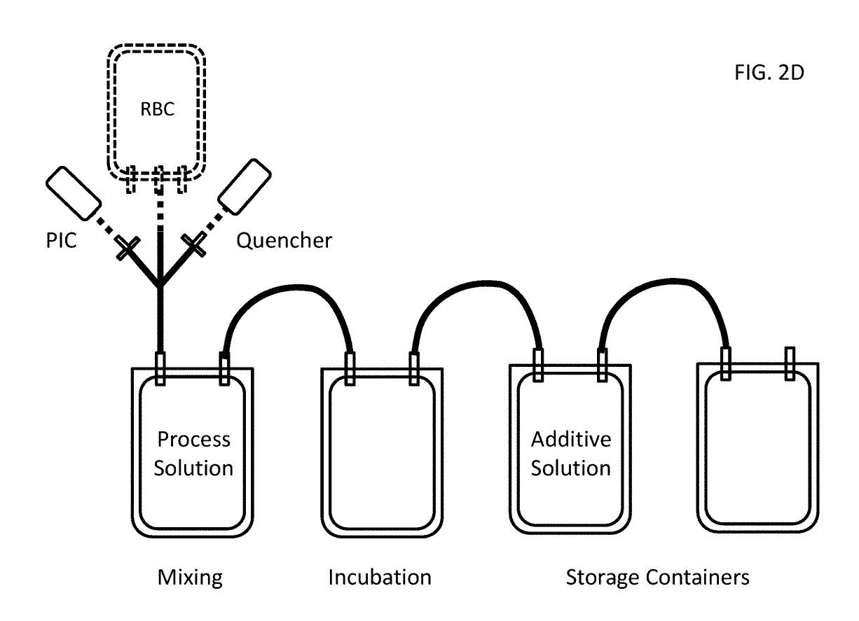

Provided herein are various processing sets and kits for use in the methods of the present disclosure (see e.g., FIGS. 1A-2D). Generally, processing sets of the present disclosure comprise suitable containers, tubing and solution(s) for introducing, mixing and/or incubating the input red blood cells, pathogen-inactivating compound, quencher and processing solution, as well as replacing the solution used during the pathogen-inactivation treatment with a red blood cell additive solution, and separating the treated red blood cells (e.g., pathogen-inactivated red blood cell preparations) into one or more (e.g., at least two) units of pathogen-inactivated red blood cells suitable for infusion into a subject. In some embodiments, a processing set is provided, comprising a) a first container (e.g., bag) suitable for receiving and mixing under sterile conditions (e.g., aseptic conditions) the red blood cells, the pathogen-inactivating compound and the quencher, wherein the first container contains a processing solution as described herein; b) a second container suitable for incubating the mixture of red blood cells, pathogen-inactivating compound, quencher and processing solution, wherein the second container is coupled (e.g., connected) to the first container such that the mixture can be transferred from the first container to the second container under sterile conditions; c) a third container suitable for replacing the solution use during the pathogen-inactivation treatment and storage of the pathogen-inactivated red blood cells, wherein the third container contains a red blood cell additive solution as described herein and is coupled (e.g., connected) to the second container such that the treated red blood cells can be transferred from the second container to the third container under sterile conditions; and optionally, d) a fourth container suitable for storage of the pathogen-inactivated red blood cells, wherein the fourth container is or may be coupled (e.g., connected) to the third container such that the pathogen-inactivated red blood cells can be transferred from the third container to the fourth container under sterile conditions. In some embodiment, the processing sets and/or kits may further comprise one or more additional containers that contain the pathogen-inactivating compound and/or quencher, wherein the additional containers are coupled or are configured to be coupled (e.g., connected) to the first container, such that the contents of the additional container(s) can be transferred to the first container under sterile conditions.

An exemplary processing set of the present disclosure is illustrated in FIG. 1A. The processing set shown in FIG. 1A comprises: a first container (e.g., bag) containing a processing solution, wherein the first container is suitable for mixing with the processing solution under sterile conditions (1) a red blood cell composition comprising a double red blood cell donation, (2) an effective amount of a pathogen-inactivating compound (PIC) (e.g., a PIC comprising a functional group which is, or which forms, a reactive electrophilic group), and (3) an effective amount of a quencher; a second container coupled (e.g., connected) to the first, wherein the second container is suitable for incubating the red blood cell composition in admixture with the processing solution, PIC and quencher under sterile conditions; and a third container containing a red blood cell additive solution, wherein the third container is coupled (e.g., connected) to the second container such that the processing solution, PIC and quencher can be replaced with a red blood cell additive solution to yield a pathogen-inactivated red blood cell preparation. In some embodiments, the third container is configured to store one unit of pathogen-inactivated red blood cells suitable for infusion into a subject. In some embodiments, the processing set further comprises a container suitable for storing the PIC, wherein the container suitable for storing the PIC is configured to be coupled with the first container such that an effective amount of the PIC can be introduced into the first container under sterile conditions. In some embodiments, the processing set further comprising a container suitable for storing the quencher, wherein the container suitable for storing the quencher is configured to be coupled with the first container such that an effective amount of the quencher can be introduced into the first container under sterile conditions. Optionally, as shown in FIG. 1A, the processing set can further include one or more storage containers. The storage container(s) are configured to be coupled (e.g., connected) with the third container, and each of the storage container(s) is configured to store one unit of pathogen-inactivated red blood cells suitable for infusion into a subject. In some embodiments, 1, 2, 3, or more storage containers are included. In some embodiments, as shown in FIG. 1B, the storage container(s) are provided with the rest of the processing set and configured to be coupled (e.g., connected) with the rest of the set (e.g., by sterile connection to the third container). In other embodiments, as shown in FIG. 1C, the storage container(s) are provided with the rest of the processing set and already coupled (e.g., connected) via sterile connection to the third container. In some embodiments, the first container is configured to be coupled (e.g., connected) with a container suitable for storing the effective amount of the PIC, a container suitable for storing the effective amount of the quencher, and/or a container suitable for storing the red blood cell composition.

Another exemplary processing set of the present disclosure is illustrated in FIG. 2A. The processing set shown in FIG. 2A comprises: a first container (e.g., bag) containing a processing solution, wherein the first container is suitable for mixing with the processing solution under sterile conditions (1) a red blood cell composition comprising a double red blood cell donation, (2) an effective amount of a pathogen-inactivating compound (PIC) (e.g., a PIC comprising a functional group which is, or which forms, a reactive electrophilic group), and (3) an effective amount of a quencher; a second container coupled (e.g., connected) to the first container, wherein the second container is suitable for incubating the red blood cell composition in admixture with the processing solution, PIC and quencher under sterile conditions; and a third container containing a red blood cell additive solution, where in the third container is coupled (e.g., connected) to the second container such that the processing solution, PIC and quencher can be replaced with a red blood cell additive solution to yield a pathogen-inactivated red blood cell preparation. In some embodiments, the third container is configured to store one unit of pathogen-inactivated red blood cells suitable for infusion into a subject. The processing set can further include sterile tubing coupled (e.g., connected) with the first container. The sterile tubing can be coupled (e.g., connected) with a container suitable for storing the effective amount of the PIC and/or a container suitable for storing the effective amount of the quencher, such that effective amounts of the PIC and the quencher can be introduced into the first container under sterile conditions. Optionally, the sterile tubing can also be coupled (e.g., connected) with a container suitable for storing the red blood cell composition, such that the red blood cell composition can be introduced into the first container under sterile conditions. In some embodiments, 1, 2, 3, or more storage containers are included. In some embodiments, as shown in FIG. 2B, the container suitable for storing the effective amount of the PIC and/or the container suitable for storing the effective amount of the quencher are provided with the rest of the processing set and configured to be coupled (e.g., connected) with the rest of the set (e.g., by sterile connection to the first container). In some embodiments, as shown in FIG. 2C, the storage container(s) are provided with the rest of the processing set and configured to be coupled (e.g., connected) with the rest of the set (e.g., by sterile connection to the third container). In other embodiments, as shown in FIG. 2D, the storage container(s) are provided with the rest of the processing set and integrated with (e.g., connected as part of) the rest of the processing set, such as for example, coupled to the third container.

In some embodiments, the present disclosure provides kits for use in the methods provided herein. In some embodiments, the kits comprise a processing set as provided herein and instruction materials that describe use of the processing set and/or kit to perform the any of the methods described herein. Kits may also contain any combination of additional components that may be necessary or useful for carrying out any of the methods described herein. Although exemplary kits are described here, the contents of other useful kits will be apparent to the skilled artisan in light of the present disclosure.

The containers as described in the processing sets and kits provided herein may be made of any material suitable and/or known in the art for processing (e.g., pathogen inactivation) and storage of red blood cells. The containers (e.g., storage containers) should allow maintenance of sterile (e.g., aseptic) conditions for its contents. The kits provided herein may further contain additive solutions, buffers, or other solutions that may be used in carrying out any of the methods provided herein.

Methods of Treatment and/or Preparation

Provided herein are methods of treating a red blood cell composition, comprising: (a) mixing (i) a red blood cell composition comprising a double red blood cell donation; (ii) an effective amount of a pathogen-inactivating compound comprising a functional group which is, or which forms, a reactive electrophilic group; (iii) an effective amount of a quencher; in a processing solution; wherein at least one of the pathogen-inactivating compound and the quencher is in (e.g., suspended in, re-suspended in) a solution comprising dextrose (e.g., about 7% to about 12% dextrose, about 10% dextrose); (b) replacing the solution used during treatment of the red blood cell composition in step (a) with a red blood cell additive solution, to yield a pathogen-inactivated red blood cell preparation; and (c) separating (e.g., transferring a portion of) the pathogen-inactivated red blood preparation of step (b) into at least two units of pathogen-inactivated red blood cells suitable for infusion into a subject (e.g., pathogen-inactivated red blood cell products suitable for infusion), wherein each unit is contained in an individual container.

Also provided herein are methods of preparing pathogen inactivated red blood cells from a double red blood cell donation, wherein the pathogen inactivated red blood cells are suitable for infusion into a subject, comprising: (a) collecting a double red blood cell donation from a donor by apheresis; (b) treating the red blood cells of the double red blood cell donation, by mixing (i) the red blood cells; (ii) an effective amount of a pathogen-inactivating compound comprising a functional group which is, or which forms, a reactive electrophilic group; (iii) an effective amount of a quencher; in a processing solution; wherein at least one of the pathogen-inactivating compound and the quencher is in a solution comprising dextrose (e.g., about 7% to about 12% dextrose, about 10% dextrose); (c) replacing the solution used during treatment of the red blood cells in step (b) with a red blood cell additive solution to yield a pathogen-inactivated red blood cell preparation; and (d) separating the pathogen-inactivated red blood cell preparation of step (c) into at least two units of pathogen-inactivated red blood cells suitable for infusion into a subject, wherein each unit is contained in an individual container.

In some embodiments, red blood cells (e.g., red blood cell composition) comprise a double donation of red blood cells (e.g., double donation of red blood cells obtained from a donor, double dose red blood cell donation; double red blood cell collection) obtained from one donor by apheresis. Apheresis collection devices/systems and methods of use thereof are well known in the art, and include for example, ALYX™ (Fenwal), AMICUS™ (Fenwal), TRIMA™ (TerumoBCT), and MCS+™ (Haemonetics).

In some embodiments, the red blood cells (e.g., red blood cell composition, red blood cells of the double red blood cell donation) comprise packed red blood cells as provided herein. In some embodiments, the red blood cells comprise a packed cell volume of about 75% to about 95% or about 80% to about 90%. In some embodiments, the red blood cell composition comprises a red cell volume of at least about 220 mL, at least about 230 mL, at least about 240 mL, at least about 250 mL, at least about 260 mL, at least about 270 mL, at least about 280 mL, at least about 290 mL, at least about 300 mL, at least about 310 mL, at least about 320 mL, at least about 330 mL, at least about 340 mL, or at least about 350 mL or more. In some embodiments, the red blood cell composition comprises a red cell volume less than or equal to 360 mL. In some embodiments, the red blood cell composition comprises a red cell volume of about 220 mL to about 400 mL, about 220 mL to about 380 mL, about 220 mL to about 360 mL, about 240 mL to about 360 mL, about 260 mL to about 360 mL, about 280 mL to about 360 mL, or about 300 mL to about 360 mL. In some embodiments, the red blood cell composition comprises a red cell volume of about 220 mL, about 230 mL, about 240 mL, about 250 mL, about 260 mL, about 270 mL, about 280 mL, about 290 mL, about 300 mL, about 310 mL, about 320 mL, about 330 mL, about 340 mL, about 350 mL, or about 360 mL. In some embodiments, the red cell volume is an absolute RBC mass volume.

In some embodiments, the red blood cell composition further comprises plasma, such as for example, residual donor plasma maintained with the red blood cells during separation from blood. In some embodiments, the red blood cell composition comprises less than about 25%, less than about 20%, less than about, 15%, less than about 10%, or less than about 5% plasma by volume (e.g., percent of total volume). In some embodiments, the red blood cell composition comprises at least about 1%, at least about 2%, at least about 3%, at least about 4%, or at least about 5% plasma by volume (e.g., percent of total volume). In some embodiments, the red blood cell composition comprises about 1% to about 25%, about 1% to about 20%, about 1% to about 15%, about 1% to about 10%, about 1% to about 5%, about 5% to about 25%, about 5% to about 20%, about 5% to about 15%, or about 5% to about 10% plasma by volume (e.g., percent of total volume). In some embodiments, the red blood cell composition further comprises an anticoagulant solution. Suitable anticoagulants are well known in the art, and may include for example, ACD or ACD-A. Suitable volumes of anticoagulants are also known in the art (e.g., about 20 mL ACD for a double dose collection, no additive solution). In some embodiments, the red blood cell composition does not contain a red blood cell additive solution. For example, the red blood cells collected and input for use in the methods provided herein may comprise packed RBCs with an amount of plasma and/or anticoagulant solution, but no other added solution or compound (e.g., no red blood additive solution) not already present in whole blood.

Effective amounts (e.g., concentration) of pathogen inactivating compound (e.g., S-303) and quencher are described in U.S. Pat. No. 8,900,805, incorporated by reference herein. In some embodiments, an effective amount of a quencher, such as for example, glutathione, in the mixture with the red blood cells and the pathogen-inactivating compound is greater than about 2 mM, greater than about 4 mM, greater than about 6 mM, greater than about 8 mM, greater than about 10 mM, greater than about 15 mM, or greater than about 20 mM. In some embodiments, the quencher concentration in the mixture is in the range of about 2 mM to 100 mM, about 2 mM to 40 mM, about 4 mM to 40 mM, about 5 mM to 40 mM, about 5 mM to 30 mM, or about 10 mM to 30 mM, or up to 2 mM, 5 mM, 10 mM, 15 mM, 20 mM, 25 mM, 30 mM, 35 mM, 40 mM, 45 mM, 50 mM, or 100 mM. In some embodiments, the quencher concentration in the mixture is about 20 mM. In some embodiments, an effective amount of a pathogen-inactivating compound, such as for example, S-303, in the mixture with the red blood cells and the quencher is in the range of about 0.05 mM to 4 mM, about 0.05 mM to 2 mM, about 0.05 mM to 0.5 mM, about 0.1 mM to 0.3 mM, or about 0.2 mM.

In some embodiments, the molar ratio of quencher to pathogen inactivation compound once both components have been mixed with the red blood cell composition is about 10:1 to about 400:1, also about 10:1 to about 200:1, also about 20:1 to about 200:1, also about 50:1 to about 200:1, also about 100:1.

In some embodiments, at least one of the pathogen-inactivating compound and the quencher is in a solution (e.g., suspended in a solution, re-suspended in a solution) comprising dextrose. The solution comprising dextrose may include, for example, dextrose at a concentration (e.g., weight/volume) of about 5% to about 15%, about 5% to about 10%, about 10% to about 15%, about 7% to about 12% dextrose, about 8% to about 12%, about 8% to about 10%, or about 5%, about 6%, about 7%, about 8%, about 9%, about 10%, about 11%, about 12%, about 13%, about 14%, or about 15% dextrose. The solution comprising dextrose may be any aqueous solution suitable for the pathogen-inactivating compound and/or quencher, and for treatment of red blood cells. In some embodiments, the solution comprising dextrose is a solution of dextrose in water or dextrose in saline, or dextrose in suitable buffer. In some embodiments, at least one of the pathogen-inactivating compound and the quencher is in a solution comprising about 7% to about 12%, about 7% to about 10%, about 5% to about 10%, or about 5%, about 6%, about 7%, about 8%, about 9%, about 10%, about 11%, or about 12% (e.g., weight/volume) dextrose in water. In some embodiments, at least one of the pathogen-inactivating compound and the quencher is in a solution comprising about 7% to about 12%, about 7% to about 10%, about 5% to about 10%, or about 5%, about 6%, about 7%, about 8%, about 9%, about 10%, about 11%, or about 12% dextrose (e.g., weight/volume) in saline. In some embodiments, only one of the pathogen-inactivating compound and the quencher is in a solution comprising dextrose (e.g., about 5-10%, about 7-10%, about 10% dextrose). In some embodiments, each of the pathogen-inactivating compound and the quencher is in a solution comprising dextrose (e.g., about 5-10%, about 7-10%, about 10% dextrose).

The quencher used in the methods described herein may be mixed with the red blood cells (RBC composition) prior to, at the same time as, or after addition of the pathogen-inactivating compound to the red blood cell composition. In some embodiments of each of the methods described herein, the quencher is added to the red blood cell composition for a suitable time interval prior to addition of the pathogen-inactivating compound, such as for example, less than about an hour, less than about 30 minutes, less than about 20 minutes, less than about 10 minutes, less than about 5 minutes, less than about 2 minutes, or less than about 1 minute before mixing the pathogen-inactivating compound with the red blood cell composition. In some embodiments, the quencher is mixed with the red blood cell composition at the same time as the pathogen-inactivating compound. If the quencher is mixed with the red blood cell composition after the pathogen-inactivating compound is mixed with the red blood cell composition, the quencher is preferably added before a significant amount of side reaction of the pathogen-inactivating compound with the red blood cells has occurred, so that adequate quenching of the undesired side reaction can be achieved. In some embodiments, the quencher is mixed with the red blood cell composition within about an hour, within about 30 minutes, within about 20 minutes, within about 10 minutes, within about 5 minutes, within about 2 minutes, or within about 1 minute after mixing the pathogen-inactivating compound with the red blood cell composition.

In some embodiments, the disclosure provides a processing solution (e.g., treatment solution, diluent solution). In some embodiments, the processing solution comprises one or more of the following components: dextrose, adenine, mannitol, citrate (e.g., sodium citrate), citric acid, and phosphate (e.g., Na₂HPO₄ and/or NaH₂PO₄). In some embodiments, the processing solution comprises one or more of adenine, mannitol, citrate, and phosphate. In some embodiments, the processing solution comprises chloride (e.g., from sodium chloride). Processing solutions (e.g., treatment solution, diluent solution) may include, for example, solutions described in U.S. Pat. No. 8,900,805. In some embodiments, the concentration of dextrose in the processing solution is from about 10 mM to about 150 mM, or about 20 mM to about 120 mM, or about 25 mM to about 100 mM, or about 30 mM to about 75 mM, or about 40 mM to about 50 mM, or about 50 mM to about 60 mM. In some embodiments, the concentration of adenine in the processing solution is from about 0.5 mM to about 5 mM, or about 0.75 mM to about 3 mM, or about 1 mM to about 2.5 mM. In some embodiments, the concentration of mannitol in the processing solution is from about 10 mM to about 150 mM, or about 20 mM to about 120 mM, or about 25 mM to about 100 mM, or about 30 mM to about 75 mM, or about 40 mM to about 60 mM. In some embodiments, the concentration of citrate (e.g., sodium citrate) in the processing solution is from about 1 mM to about 100 mM, or about 2 mM to about 75 mM, or about 5 mM to about 50 mM, or about 7.5 mM to about 25 mM, or about 10 mM to about 15 mM. In some embodiments, the concentration of phosphate (e.g., Na₂HPO₄ and/or NaH₂PO₄) in the processing solution is from about 1 mM to about 150 mM, or about 2 mM to about 100 mM, or about 3 mM to about 75 mM, or about 4 mM to about 50 mM, or about 5 mM to about 25 mM, or about 10 mM to about 20 mM. In some embodiments, the concentration of chloride in the processing solution is from about 250 mM, or about 200 mM, or about 150 mM, or about 120 mM, or about 100 mM, or about 90 mM, or about 80 mM, or about 70 mM, or about 60 mM, or about 50 mM, or about 40 mM, or about 30 mM, or about 20 mM, about 10 mM, or about 25 to about 250 mM, or about 40 to about 100 mM, or about 50 to about 75 mM, or about 60 to about 70 mM, or about 100 to about 200 mM, or about 125 mM to about 175 mM. In some embodiments, the processing solution comprises 10 mM to about 150 mM (or about 35 mM to about 65 mM) dextrose, 0.5 mM to about 5 mM (or about 0.75 mM to about 3 mM) adenine, about 10 mM to about 150 mM (or about 25 mM to about 75 mM) mannitol, about 5 mM to about 75 mM (or about 10 mM to about 20 mM) citrate (e.g., sodium citrate), about 3 mM to about 75 mM (or about 5 mM to about 25 mM) phosphate (e.g., Na₂HPO₄ and/or NaH₂PO₄), and about 5 to about 100 mM, or (about 25 to about 75 mM) chloride.

In some embodiments, methods of the disclosure also provides for incubating the mixture of red blood cells, pathogen-inactivating compound, quencher and processing solution, prior to the replacement of solution used during treatment. The incubation may comprise, for example, the period of time between the point of addition of the pathogen inactivating-compound and quencher to the point of replacing the solution used during treatment. In some embodiments, the mixture is incubated in a temperature range of about 1° C. to 30° C., about 18° C. to 25° C., about 20° C. to about 25° C., about 37° C., or about room temperature. In some embodiments, the mixture is incubated for at least 2 hours, at least 6 hours, at least 12 hours, or at least 18 hours. In some embodiments, the mixture is incubated for 24 hours or less. In some embodiments, the mixture is incubated for about 2 hours to about 24 hours, about 6 hours to about 24 hours, about 12 hours to about 24 hours, or about 18 hours to about 24 hours.

The disclosed methods also provide for replacement of the solution used during pathogen inactivation treatment of the red blood cells, such as for example after the incubation period described above, to yield a pathogen-inactivated red blood cell preparation. Any suitable red blood cell additive solution may be used for replacement, and several such additive solutions known in the art have been approved by one or more regulatory agencies or accrediting organizations. Exemplary red blood cell additive solutions include, but are not limited to, those set forth in the following Table 1.

TABLE 1
Red blood cell additive solutions

| | | | | Eryth- | | PAGGS- | |
|---|---|---|---|---|---|---|---|
| | AS-1 | AS-3 | SAG-M | rosol | AS-5 | M | MAP |
| Dextrose | 111.0 | 55.5 | 45.4 | 81.1 | 45.4 | 47.5 | 36.4 |
| Adenine | 2.0 | 2.2 | 1.3 | 1.6 | 2.2 | 1.4 | 1 |
| Guanosine | | | | | | 1.44 | |
| Mannitol | 41.2 | | 28.8 | 42.5 | 28.8 | 55 | 80 |
| Sodium | | 20 | | 26.6 | | | 5.1 |
| Citrate | | | | | | | |
| Na₂HPO₄ | | | | 17 | | 8 | |
| NaH₂PO₄ | | 20 | | 4.7 | | 8 | 6 |
| NaCl | 154.0 | 70 | 150 | | 150 | 72 | 85 |
| Citric Acid | | 2 | | | | | 1 |
| Osmolality | | 276 | 359 | 175 | 351 | 296 | |
| (mOsm) | | | | | | | |

Components indicated as mM concentrations

Following treatment of red blood cells, such as for example, by mixing of RBCs with an effective amount of a pathogen-inactivating compound and an effective amount of a quencher, in a processing solution, as provided herein, and replacing the solution used during treatment with a red blood cell additive solution, the pathogen-inactivated red blood cell preparation is separated (e.g., transferred) into containers to provide at least two units of pathogen-inactivated red blood cells suitable for infusion into a subject. Transfer of the pathogen-inactivated red blood cell preparation into individual unit containers is generally a manual process (e.g., gravity flow) and the at least two units of pathogen-inactivated red blood cells are preferably adjusted to approximately equivalent volumes (e.g., unit weight). The individual unit containers then may be disconnected from the processing set, and each other, by using suitable tube sealing devices known in the art.

Units of pathogen-inactivated red blood cells may be stored, prior to infusion into a subject. In some embodiments, the units of pathogen-inactivated red blood cells may be stored at refrigerated temperature, such as for example at 1° C. to 6° C. or 2° C. to 4° C. the units of pathogen-inactivated red blood cells may be stored at least about 1 day, about 2 days, about 3 days, about 4 days, about 5 days, about 6 days, about 7 days, 10 days, about 14 days, about 21 days, about 28 days, about 35 days, or about 42 days or more. In some embodiments, the units of pathogen-inactivated red blood cells may be stored for up to about 42 days.

### Methods of Providing an Infusion

In another aspect, the present disclosure also provides methods of infusing red blood cells into a subject in need thereof, comprising infusing into the subject pathogen-inactivated red blood cells as described herein, such as for example a unit of pathogen-inactivated red blood cells (e.g., therapeutic dosage unit) prepared according to any of the disclosed methods. Red blood cell infusions for a variety of medical indications are known in the art, and pathogen-inactivated red blood cells of the present disclosure may be used for infusion in a similar manner as non-pathogen inactivated (e.g., conventional) red blood cells.

The present disclosure is further illustrated by the following non-limiting examples.

### EXAMPLES

### Example 1: Evaluation of Packed Apheresis RBCs in Reconstitution Solutions

To evaluate alternative reconstitution solutions for possible use with an S-303 (amustaline dihydrochloride) pathogen-inactivating compound and/or glutathione (GSH) quencher in treating packed apheresis RBCs (paRBC) without additive solution, GSH was reconstituted with one of four solutions: 0.9% normal saline, 10% dextrose in water (i.e., 10.0 g dextrose dissolved in 100 mL WFI), 5% dextrose in 0.9% saline (i.e., 5 g dextrose dissolved in 100 mL 0.9% saline) and 5% dextrose in 0.45% saline (i.e., 5.0 g dextrose dissolved in 50 mL 0.9% saline+50 mL WFI). For each sample, 18 mL of apheresis RBCs were mixed with a mixture of 7 mL diluent (INTERCEPT® Blood System for RBCs) and 0.75 mL of 600 mM GSH (final concentration 17.5 mM) reconstituted with the above solutions. The samples were incubated and evaluated for osmolarity using an Osmometer 3250 (Advanced Instruments), percent hemolysis by measuring hemoglobin in cell free supernatants using an Advia Hematology Analyzer (Siemens), and glucose concentration using an ABX Pentra C200 Chemistry Analyzer (Horiba Medical).

Glucose concentrations for each reconstitution condition were measured as 8.0 mmol/L for the RBCs with GSH reconstituted in 0.9% saline, 22.4 mmol/L for the RBCs with GSH reconstituted in 10% dextrose in water, 17.4 mmol/L for the RBCs with GSH reconstituted in 5% dextrose in 0.9% saline and 17.3 mmol/L for the RBCs with GSH reconstituted in 5% dextrose in 0.45% saline. For reference, the average glucose concentration across multiple untreated apheresis RBC units was 29.9 mmol/L.

Percent hemolysis for each reconstitution condition was measured as 0.077 for the RBCs with GSH reconstituted in 0.9% saline, 0.075 for the RBCs with GSH reconstituted in 10% dextrose in water, 0.101 for the RBCs with GSH reconstituted in 5% dextrose in 0.9% saline and 0.179 for the RBCs with GSH reconstituted in 5% dextrose in 0.45% saline.

Average osmolarity for each reconstitution condition was measured as 290 (0.6 SD) mOsm for the RBCs with GSH reconstituted in 0.9% saline, 296 (1.0 SD) mOsm for the RBCs with GSH reconstituted in 10% dextrose in water, 305 (8.7 SD) mOsm for the RBCs with GSH reconstituted in 5% dextrose in 0.9% saline and 292 (2.1 SD) mOsm for the RBCs with GSH reconstituted in 5% dextrose in 0.45% saline.

Relative to each other, GSH reconstituted in 10% dextrose in water provided the best hemolysis and glucose results and second best osmolarity result. GSH reconstituted in 5% dextrose in 0.9% saline provided the best osmolarity result and second best results for both hemolysis and glucose. This evaluation indicted a benefit of reconstitution of GSH in dextrose, and particularly reconstitution in a solution of 10% dextrose in water.

### Example 2: Comparison of S-303 Reconstitution Solutions for Packed Apheresis RBCs

Packed apheresis red blood cells (paRBC) were divided into two pathogen inactivation treatment arms and mixed for treatment with the quencher GSH, followed by the pathogen inactivation compound S-303 reconstituted in either 0.9% saline or 10% dextrose in water. The GSH for both arms was reconstituted in a solution of 10% dextrose in water. Following treatment incubation at 22° C., various metabolic and physical parameters were measured for the red blood cells at days 2, 14 and 35, including for example, pH, ATP, extracellular K+, Na+, glucose and lactate, hemolysis, hemoglobin (Hb), hematocrit (Hct) and morphology. Analytical methods were performed using standard instrumentation and methods known in the art, such as for example, blood gas analyzer, K+/Na+analyzer, chemistry analyzer, hematology analyzer, and spectrophotometric measurements. Data are shown in Table 2.

TABLE 2
RBC parameters pre- and post-treatment

| | | D 2 | | D 14 | | D 35 | |
|---|---|---|---|---|---|---|---|
| | Pre | R1 | R2 | R1 | R2 | R1 | R2 |
| pH @ 37° C. | 6.9 | 6.8 | 6.8 | 6.6 | 6.6 | 6.4 | 6.5 |
| pH @ 22° C. | 7.2 | 7.0 | 7.0 | 6.8 | 6.8 | 6.7 | 6.7 |
| K+ (mmol/L) | 2.7 | 1.9 | 2.0 | 22.1 | 22.4 | 42.4 | 40.5 |
| Na+ (mmol/L) | 157.0 | 139.7 | 137.9 | 122.7 | 120.4 | 110.2 | 111.2 |
| ATP (umol/gHb | 4.0 | 5.7 | 5.4 | 5.6 | 5.2 | 3.4 | 3.2 |
| Glucose (mmol/L) | 6.5 | 28.7 | 37.1 | 28.9 | 34.2 | 20.3 | 29.4 |
| Lactate (mmol/L) | 1.9 | 7.8 | 8.0 | 17.2 | 16.9 | 23.0 | 22.7 |
| Hemolysis (%) | 0.01 | 0.04 | 0.02 | 0.08 | 0.09 | 0.21 | 0.18 |
| Hb (g/dL) | 26.8 | 18.4 | 18.2 | 18.3 | 17.8 | 18.1 | 17.6 |
| Hct (%) | 86.4 | 60.7 | 61.6 | 62.5 | 59.9 | 62.0 | 61.0 |

Among the parameters measured, RBCs treated with S-303 and GSH that were both reconstituted with 10% dextrose in water showed significantly higher extracellular glucose at each time point.

### Example 3: Pathogen Inactivation of Double-Apheresis RBCs Collected in ACD

A processing set for pathogen inactivation of double apheresis RBCs is prepared as configured in FIG. 2d. The processing set is configured to include a fourth container (e.g., storage container), as well as a trifurcated lead that is configured to be coupled to containers that contain the input RBCs (packed, double apheresis RBCs with ACD anticoagulant and plasma), pathogen-inactivating compound S-303 (amustaline dihydrochloride) and quencher glutathione (GSH) sodium salt. The first container contains 140 mL of processing solution and the third container contains 180 mL of SAG-M red blood cell additive solution. Alternatively, if desired, the third and fourth containers may each contain half of the additive solution.

For treating the double apheresis RBCs, the glutathione sodium salt quencher (GSH) is re-suspended (e.g., re-constituted) in 10% dextrose in water solution, and each of the RBCs (e.g., 220-360 mL) and reconstituted quencher containers are coupled to the processing set via the trifurcated lead, with the RBCs and GSH subsequently transferred into the first container and mixed with processing solution. Next, the amustaline dihydrochloride PIC is re-suspended in 10% dextrose in water solution, and reconstituted PIC is transferred into the first container to mix with the RBCs and quencher, to achieve a concentration in the mixture of about 20 mM GSH and about 0.2 mM amustaline, based on a nominal RBC input volume of 280 mL. The mixture is then transferred to the second container and incubated for 18-24 hours at 20-25° C. Following the incubation, the processing set with the mixture is centrifuged to concentrate the RBCs, and the supernatant expressed back into the first container, which is then sealed and detached from the rest of the processing set. The 180 mL of SAG-M red blood cell additive solution in the third container is transferred into the second container, the pathogen-inactivated RBC concentrate resuspended and the contents transferred and distributed approximately equally into the third and fourth containers (e.g., storage bags) for storage until infusion. These two containers are then sealed and detached from the processing set. Pathogen inactivated RBCs are characterized to ensure suitable quality by standard methods known in the art (see e.g., Example 2).

What is claimed is:

1. A method of treating a red blood cell composition, comprising:
(a) mixing
(i) a red blood cell composition comprising a red cell volume of at least about 220 mL;
(ii) an effective amount of a pathogen-inactivating compound comprising a functional group which is, or which forms, a reactive electrophilic group; and
(iii) an effective amount of a quencher;
in a processing solution;
wherein at least one of the pathogen-inactivating compound and the quencher is in a solution comprising about 5% to about 12% dextrose;
(b) replacing the solution used during treatment of the red blood cell composition in step (a) with a red blood cell additive solution, to yield a pathogen-inactivated red blood cell preparation; and
(c) separating the pathogen-inactivated red blood preparation of step (b) into at least two units of pathogen-inactivated red blood cells suitable for infusion into a subject, wherein each unit is contained in an individual container.

2. The method of claim 1, wherein the red blood cell composition comprises a double red blood cell donation.

3. The method of claim 2, wherein the double red blood cell donation is obtained from a donor by apheresis.

4. The method of any one of claims 1-3, wherein the red blood cell composition comprises packed red blood cells.

5. The method of any one of claims 1-4, wherein the red blood cell composition comprises a packed cell volume of about 75% to about 95%.

6. The method of claim 5, wherein the red blood cell composition comprises a packed cell volume of about 80% to about 90%.

7. The method of any one of claims 1-6, wherein the red blood cell composition comprises a red cell volume of about 220 mL to about 420 mL.

8. The method of any one of claims 1-7, wherein the red blood cell composition further comprises plasma.

9. The method of claim 8, wherein the red blood cell composition comprises less than about 5% plasma by volume.

10. The method of any one of claims 1-9, wherein the red blood cell composition further comprises an anticoagulant solution.

11. The method of any one of claims 1-10, wherein the red blood cell composition does not contain a red blood cell additive solution.

12. The method of any one of claims 1-11, wherein the pathogen-inactivating compound comprises a nucleic acid binding ligand that is an intercalator.

13. The method of claim 12, wherein the intercalator is an acridine.

14. The method of any one of claims 1-13 wherein the pathogen-inactivating compound is (3-alanine, N-(acridin-9-yl), 2-[bis(2-chloroethyl)amino]ethyl ester.

15. The method of any one of claims 1-14, wherein the quencher comprises cysteine or a derivative of cysteine.

16. The method of any one of claims 1-14, wherein the quencher is a peptide of 3-6 amino acids, wherein at least one of the amino acids is cysteine, N-acetyl cysteine, or S-acetyl cysteine.

17. The method of any one of claims 1-16, wherein the quencher is glutathione or a pharmaceutically acceptable salt thereof.

18. The method of claim 17, wherein the quencher is glutathione monosodium salt.

19. The method of any one of claims 1-18, wherein at least one of the pathogen-inactivating compound and the quencher is in a solution comprising 7-12% dextrose in water.

20. The method of any one of claims 1-18, wherein at least one of the pathogen-inactivating compound and the quencher is in a solution comprising 7-12% dextrose in saline.

21. The method of any one of claims 1-18, wherein only one of the pathogen-inactivating compound and the quencher is in a solution comprising 7-12% dextrose.

22. The method of any one of claims 1-18, wherein each of the pathogen-inactivating compound and the quencher is in a solution comprising 7-12% dextrose.

23. The method of any one of claims 1-22, wherein the processing solution comprises one or more of dextrose, adenine, mannitol, citrate, citric acid, and phosphate.

24. The method of claim 23, wherein the processing solution comprises one or more of adenine, mannitol, citrate, and phosphate.

25. The method of any one of claims 1-24, further comprising incubating the mixture of step (a) prior to the replacement of solution in step (b).

26. The method of any one of claims 1-25, wherein the pathogen-inactivated red blood cell composition of step (b) is separated into two units of pathogen-inactivated red blood cells suitable for infusion into a subject.

27. The method of any one of claims 1-26, wherein the at least two units of pathogen-inactivated red blood cells suitable for infusion into a subject each comprise a therapeutic dosage unit of red blood cells.

28. The method of any one of claims 1-27, further comprising storing the at least two units of pathogen-inactivated red blood cells at refrigerated temperature.

29. A unit of pathogen-inactivated red blood cells suitable for infusion into a subject prepared by the method of any one of claims 1-26.

30. A method of preparing pathogen inactivated red blood cells from a double red blood cell donation, wherein the pathogen inactivated red blood cells are suitable for infusion into a subject, comprising:
(a) collecting a double red blood cell donation from a donor by apheresis;
(b) treating the red blood cells of the double red blood cell donation, by mixing
(i) the red blood cells;
(ii) an effective amount of a pathogen-inactivating compound comprising a functional group which is, or which forms, a reactive electrophilic group; and
(iii) an effective amount of a quencher;
in a processing solution;
wherein at least one of the pathogen-inactivating compound and the quencher is in a solution comprising about 5% to about 12% dextrose;
(c) replacing the solution used during treatment of the red blood cells in step (b) with a red blood cell additive solution to yield a pathogen-inactivated red blood cell preparation; and
(d) separating the pathogen-inactivated red blood cell preparation of step (c) into at least two units of pathogen-inactivated red blood cells suitable for infusion into a subject, wherein each unit is contained in an individual container.

31. The method of claim 30, wherein the red blood cells comprise packed red blood cells.

32. The method of claim 30 or claim 31, wherein the red blood cells comprise a packed cell volume of about 75% to about 95%.

33. The method of claim 32, wherein the red blood cells comprise a packed cell volume of about 80% to about 90%.

34. The method of any one of claims 30-33, wherein the red blood cells comprise a red cell volume of at least about 220 mL.

35. The method of any one of claims 30-33, wherein the red blood cells comprise a red cell volume of about 220 mL to about 420 mL.

36. The method of any one of claims 30-35, wherein the red blood cells further comprise plasma.

37. The method of claim 36, wherein the red blood cells comprise less than about 5% plasma by volume.

38. The method of any one of claims 30-37, wherein the red blood cells further comprise an anticoagulant solution.

39. The method of any one of claims 30-38, wherein the red blood cells do not contain a red blood cell additive solution.

40. The method of any one of claims 30-39, wherein the pathogen-inactivating compound comprises a nucleic acid binding ligand that is an intercalator.

41. The method of claim 40, wherein the intercalator is an acridine.

42. The method of any one of claims 30-41 wherein the pathogen-inactivating compound is β-alanine, N-(acridin-9-yl), 2-[bis(2-chloroethyl)amino]ethyl ester.

43. The method of any one of claims 30-42, wherein the quencher comprises cysteine or a derivative of cysteine.

44. The method of any one of claims 30-42, wherein the quencher is a peptide of 3-6 amino acids, wherein at least one of the amino acids is cysteine, N-acetyl cysteine, or S-acetyl cysteine.

45. The method of any one of claims 30-44, wherein the quencher is glutathione or a pharmaceutically acceptable salt thereof.

46. The method of claim 45, wherein the quencher is glutathione monosodium salt.

47. The method of any one of claims 30-46, wherein at least one of the pathogen-inactivating compound and the quencher is in a solution comprising 7-12% dextrose in water.

48. The method of any one of claims 30-46, wherein at least one of the pathogen-inactivating compound and the quencher is in a solution comprising 7-12% dextrose in saline.

49. The method of any one of claims 30-48, wherein only one of the pathogen-inactivating compound and the quencher is in a solution comprising 7-12% dextrose.

50. The method of any one of claims 30-48, wherein each of the pathogen-inactivating compound and the quencher is in a solution comprising 7-12% dextrose.

51. The method of any one of claims 30-50, wherein the processing solution comprises one or more of dextrose, adenine, mannitol, citrate, citric acid, and phosphate.

52. The method of claim 51, wherein the processing solution comprises one or more of adenine, mannitol, citrate, and phosphate.

53. The method of any one of claims 30-52, further comprising incubating the mixture of step (b) prior to the replacement of solution in step (c).

54. The method of any one of claims 30-53, wherein the pathogen-inactivated red blood composition of step (b) is separated into two units of pathogen-inactivated red blood cells suitable for infusion into a subject.

55. The method of any one of claims 30-54, wherein the at least two units of pathogen-inactivated red blood cells suitable for infusion into a subject each comprise a therapeutic dosage unit of red blood cells.

56. The method of any one of claims 30-55, further comprising storing the at least two units of pathogen-inactivated red blood cells at refrigerated temperature.

57. A unit of pathogen-inactivated red blood cells suitable for infusion into a subject prepared by the method of any one of claims 30-56.

58. A method of infusing red blood cells into a subject in need thereof, comprising infusing into the subject a unit of pathogen-inactivated red blood cells of claim 29 or claim 57.

59. A processing set for preparing pathogen inactivated red blood cells from a double red blood cell donation, wherein the pathogen inactivated red blood cells are suitable for infusion into a subject, the set comprising:
(a) a first container containing a processing solution, wherein the first container is suitable for mixing with the processing solution under sterile conditions (1) a red blood cell composition comprising a double red blood cell donation, (2) an effective amount of a pathogen-inactivating compound (PIC), and (3) an effective amount of a quencher;
(b) a second container coupled to the first container, wherein the second container is suitable for incubating the red blood cell composition in admixture with the processing solution, PIC and quencher under sterile conditions;
(c) a third container containing a red blood cell additive solution, where in the third container is coupled to the second container such that the processing solution, PIC and quencher can be replaced with a red blood cell additive solution to yield a pathogen-inactivated red blood cell preparation, and wherein the third container is configured to store one unit of pathogen-inactivated red blood cells suitable for infusion into a subject; and
(d) one or more storage containers configured to be coupled with the third container, wherein each of the one or more storage containers is configured to store one unit of pathogen-inactivated red blood cells suitable for infusion into a subject.

60. The processing set of claim 59, further comprising a container suitable for storing the PIC, wherein the container suitable for storing the PIC is configured to be coupled with the first container such that an effective amount of the PIC can be introduced into the first container under sterile conditions.

61. The processing set of claim 59 or 60, further comprising a container suitable for storing the quencher, wherein the container suitable for storing the quencher is configured to be coupled with the first container such that an effective amount of the quencher can be introduced into the first container under sterile conditions.

62. The processing set of any one of claims 59-61, wherein the processing set comprises a storage container coupled to the third container, wherein each of the third container and the storage container is configured to store one unit of pathogen-inactivated red blood cells suitable for infusion into a subject.
